(19) United States
(12) Patent Application Publication
(10) Pub. No.: US 2005/0154338 A1
(43) Pub. Date: Jul. 14, 2005

(54) Brace 2000

(76) Inventor: Angel Gerena, Asbury Park, NJ (US)

Correspondence Address: Angel Gerena, 905 Emery St. - Apt. #2, Asbury Park, NJ, 07712, US

(21) Appl. No.: 10/757,732

(22) Filed: Jan. 14, 2004

(51) Int. Cl. A61F 5/00

(52) U.S. Cl. 602/21

(57) ABSTRACT

This new wrist brace support device has two rigid splints that will add extra support and protection to the user like no other wrist brace support device.

### FIELD OF THE INVENTION

The invention relates in general to wrist brace support devices in particular to more modern wrist brace support devices.

### BRIEF DESCRIPTION OF THE DRAWINGS

FIG. 1 shows you a wrist brace support device with 3 velcro straps and two rigid splint pockets.

FIG. 2 shows you a wrist brace support device with 3 velcro straps two rigid splint pockets and two rigid splints one rigid splint for each pocket.

FIG. 3 shows you a wrist brace support device from the side.

### DESCRIPTION OF THE PREFERRED EMBODIMENT

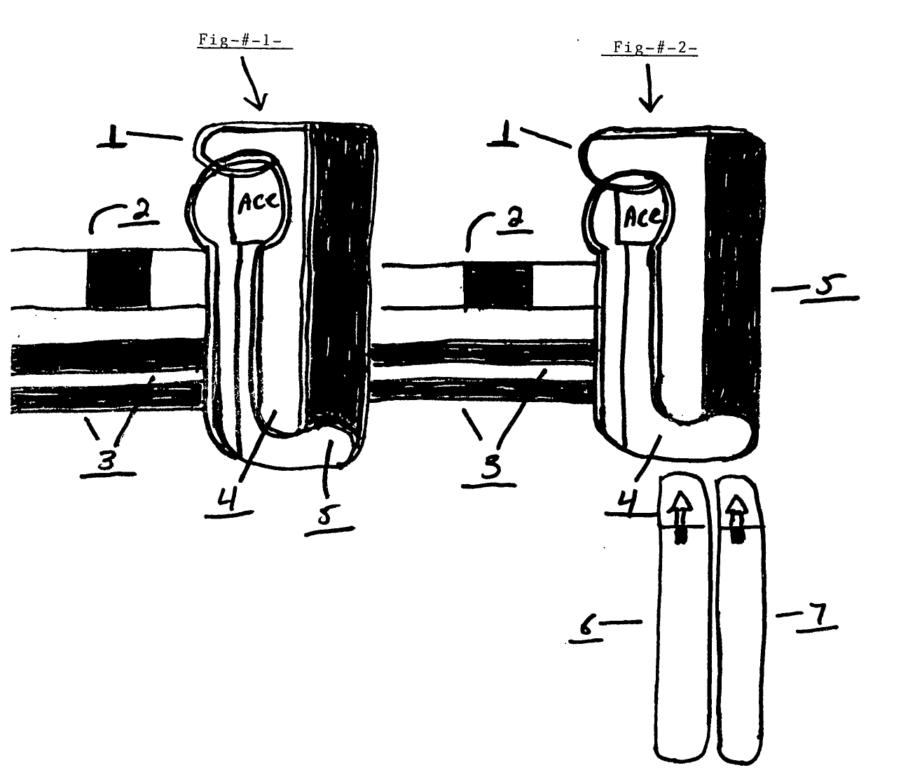

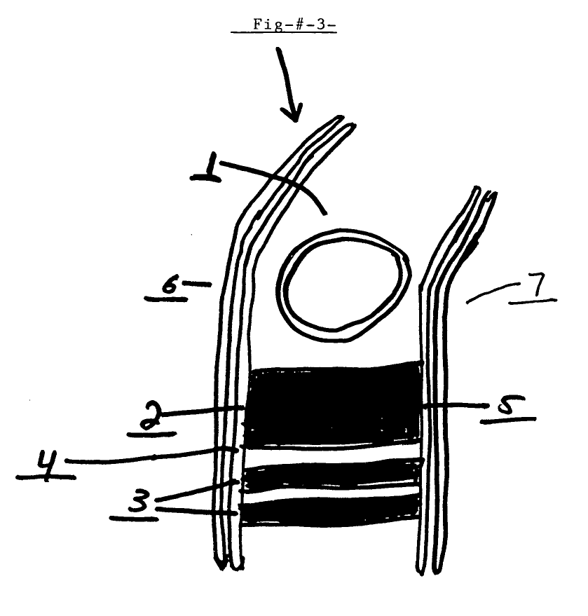

Reffering first to FIG. 1 the illustration is a wrist brace suppoert device.with two pockets and 3 velcro straps for support. FIG. 2 shows you a wrist support device that has two pockets and two rigid splints one splint fits in the inside of the brace and the other splint fits on the outside of the braceadding extra support to the person using it.

This document is a patent application for a wrist support device with two rigid splints that add extra support and protection to the user with one rigid splint in the inside pocket of wrist brace support device and one splint on the outside pocket of the wrist brace.

### DETAILED DESCRIPTION OF THE INVENTION

FIG. 1 is a rigid splint that is placed on the outside pocket of the wrist brace support device,

That adds extra support and protection to the user.

### DETAILED DESCRIPTION OF THE INVENTION

FIG. 2 is a rigid splint according to claim 1 wherein said is an item that is hard and solid like stiffly stiffen stiffness compact substantial solidly solidity solidify.

FIG. 3 is a rigid splint according to claim 2 wherein said is stiff rigidly splinter.

FIG. 4 is a rigid splint according to claim 3 wherein said has the shape of the users hand.

FIG. 5 is a rigid splint according to claim 4 wherein said external form or the users hand.

FIG. 6 is a rigid splint according to claim 5 wherein said external form or the appearance mold or pattern give shape to mold.

FIG. 7 is a rigid splint.

FIG. 8 is the wrist support of claim 7 wherein the proximal portion of the member is curved upwards at a 30 degree angle.

FIG. 9 is a rigid splint according to claim 8 is something hard firm not soft dificult severe indisputable harden hardness.

### DETAILED DESCRIPTION OF THE INVENTION

This new wrist support device has two rigid splints one for the outside pocket of the wrist brace support device that will provide extra support to the person using it from the inside and from the outside of the wrist.

FIG. 1 it shows you a wrist brace with a rigid splint number one is the web FIG. 2 is the long strap number 3 are the velcro straps number 4 is the pocket for the rigid splint and number 5 is a pocket for a rigid splint.

FIG. 2 shows you a wrist support device that has two pockets and two rigid splints one rigid splint fits in the outside pocket of the wrist brace adding extra support to the person using it.

1 is the web number 2 is the long strap and number 3 are the velcro straps number 4 and 5 are the pockets

FIG. 2 shows you two rigid splints number 6 fits in the inside of the brace.

And number 7 is a rigid splint that fits in the outside pocket of the wrist brace.

### DETAILED DESCRIPTION OF THE INVENTION

FIG. 2 shows you the wrist brace with double rigid splints and how they are located number 1 is the web

number 2 is the long strap number 3 are the velcro straps number 4 is the pocket for the rigid splint and 5 is a pocket

number 6 is the rigid splint and 7 is a rigid splint.

### DETAILED DESCRIPTION OF THE INVENTION

FIG. 3 shows you the wrist brace with double rigid splints and how they are located number 1 is the web number 2 is the long strap number 3 are the two velcros number 4 is one of the pockets number 5 is the other pocket number 6 is the rigid splint that fits in the inside of the brace number 7 is the rigid splint that fits in the outside pocket of the brace.

the rigid splint in the outside pocket provides the user with the extra protection and support without taking off the wrist brace.

the user may sign any document or do other normal things without taking off the wrist brace.

1) A rigid splint that is placed on the outside pocket of the wrist brace support device comprising.

2) A rigid splint according to claim 1 wherein said is an item that is hard and solid like stiffly stiffen stiffness compact substantial solidly solidity solidify comprising.

3) A rigid splint according to claim 2 wherein said is stiff rigidly splinter comprising.

4) A rigid splint according to claim 3 wherein said has the shape of the users hand comprising.

5) A rigid splint according to claim 4 wherein said external form or the users hand comprising.

6) A rigid splint according to claim 5 wherein said external form or appearance mold or pattern give shape to mold comprising.

7) A rigid splint comprising.

8) the wrist support of claim 7 wherein the proximal portion of the member is curved upwards at about a 30 degree angle. comprising.

9) A rigid splint according to claim 8 is something hard firm not soft dificult severe disputable harden comprising.
